(19) United States
(12) Patent Application Publication
(10) Pub. No.: US 2007/0167804 A1
(43) Pub. Date: Jul. 19, 2007

(54) Tubular compliant mechanisms for ultrasonic imaging systems and intravascular interventional devices

(76) Inventors: Byong-Ho Park, Cincinnati, OH (US); Stephen M. Rudy, Palo Alto, CA (US); Friedrich B. Prinz, Woodside, CA (US); David H. Liang, Menlo Park, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/510,134

(22) Filed: Aug. 24, 2006

Related U.S. Application Data
(63) Continuation-in-part of application No. 10/667,230, filed on Sep. 18, 2003, now Pat. No. 7,115,092.
(60) Provisional application No. 60/411,924, filed on Sep. 18, 2002.
(60) Provisional application No. 60/711,654, filed on Aug. 25, 2005.

(51) Int. Cl. A61B 1/00

(52) U.S. Cl. 600/459; 600/143

(57) ABSTRACT

A micromanipulator comprising a tubular structure and a structural compliance mechanism that are formed from a tube made of an elastic and/or superelastic material. The micromanipulator is useful for intravascular interventional applications and particularly ultrasonic imaging when coupled with an ultrasound transducer. Also disclosed are medical devices for crossing vascular occlusions using radio-frequency energy or rotary cutting, preferably under the guidance of real-time imaging of the occlusion, as well as accompanying methods.

### CROSS-REFERENCE TO RELATED APPLICATIONS

This application is a continuation-in-part of U.S. patent application Ser. No. 10/667,230 filed on Sep. 18, 2003 which claims the benefit of U.S. Provisional Patent Application No. 60/411,924, filed Sep. 18, 2002; and this application also claims the benefit of U.S. Provisional Application No. 60/711,654, filed on Aug. 25, 2005; the entire contents and appendices of which are hereby incorporated by reference.

### BACKGROUND OF THE INVENTION

1. Field of the Invention

The present invention relates generally to medical devices and methods for intravascular imaging combined with therapeutic devices to cross a partially or completely occluded blood vessel.

2. Description of the Related Art

Currently, heart disease such as heart attack and stroke is the number one killer in the United States. One out of four men and women would experience this disease during his/her lifetime. In this category, the coronary artery disease is the most serious and often requires an emergency operation to save lives. The main cause of the coronary artery disease is the accumulation of plaques inside an artery, which forms on the lumen walls of a blood vessel and restricts blood flow. In some instances, the extent of occlusion of the lumen is so severe that the lumen is completely or nearly completely obstructed. Such a condition may be described as a total occlusion. If this occlusion persists for a long period of time, the lesion is referred to as a chronic total occlusion or CTO. These occlusions may consist of soft atheroma or tougher, fibrotic occlusions.

Several solutions are available, e.g., balloon angioplasty, rotational atherectomy, and intravascular stents (balloon-expandable wire mesh implants), to open up the clogged section, which is called stenosis. In order to place the distal end of a catheter used in such treatments at the site of the occlusion, a guidewire is typically introduced into the patient's vasculature at an incision removed from the site of treatment, often in the femoral artery. The guidewire is then threaded through the patient's vascular system to the site of the occlusion, where it is pushed through the stenosis, and across the lesion. Then, a catheter, for example a balloon catheter, can be inserted over the guidewire and directed to the treatment site along the guidewire. Ordinarily, the distal end of the guidewire is quite flexible so that as it is pushed through the vasculature, it can find its way through the turns of the typically irregular passageway without damaging the vasculature.

In some instances, the extensive plaque formation of a chronic total occlusion may be calcified and difficult to penetrate with a conventional guidewire. If the guidewire cannot cross the occlusion, the therapeutic catheter cannot be properly placed in the lesion, and the therapeutic intervention, such as balloon angioplasty or stent placement, cannot be carried out. In such cases, a recanalizing device such as a stiffer guidewire may be employed to cross the stenosis. In such cases, additional precautions must be taken to prevent injury to the vessel wall. For example, it is imperative that the guidewire or other recanalizing device be centered within the vessel to avoid penetration of the vessel wall.

Traditionally, during the operation, surgeons rely on X-ray fluoroscopic images that are basically planary images showing the external shape of the silhouette of the lumen of blood vessels. Unfortunately, with X-ray fluoroscopic images, there is a great deal of uncertainty about the exact extent and orientation of the atherosclerotic lesions responsible for the occlusion to find the exact location of the stenosis. In addition, though it is known that restenosis can occur at the same place, it is difficult to check the condition inside the vessels after surgery.

In order to resolve these issues, an ultrasonic transducer has been implemented in the endovascular intervention to visualize the inside of the blood vessels. To date, however, the ultrasonic transducer is only able to see side images of the blood vessels by rotating the transducers in parallel to the blood vessels. Thus, known ultrasonic transducers have a fundamental limitation in their uses in endovascular/intravascular applications. There remains a need for improved medical devices which enhance visualization of blood vessels and assist in crossing difficult vascular occlusions.

### SUMMARY OF THE INVENTION

The present invention addresses this need in the art by disclosing a new micromanipulator useful for ultrasonic imaging, intravascular intervention, and the like. The micromanipulator enables its user to visualize and inspect inside blood vessels in essentially all directions and to treat any abnormalities identified in a minimally invasive manner. In one embodiment, intravascular ultrasound (IVUS) imaging using the disclosed micromanipulator is combined with various therapeutic devices. This combined device can be used to treat a diseased vessel under the guidance of intravascular imaging. This novel combination device, therapeutic device plus intravascular imaging, is able to successfully penetrate and recanalize vasculature with highly calcified or hard plaques.

In a preferred embodiment, the therapeutic device is a guidewire adapted to cross lesions with the assistance of radio-frequency (RF) energy emitted from the tip of the guidewire. In this embodiment, the guidewire is similar to standard guidewires, and can be placed adjacent to the lesion using standard X-ray fluoroscopic images. Once in place, a catheter including a means to visualize the area in front of the catheter, preferably IVUS, is placed over the guidewire and advanced through the vasculature to the point of the lesion. The guidewire is then advanced through the lesion under the visual guidance provided by the catheter. If needed, RF energy can be used to facilitate crossing the lesion.

In another embodiment, the guidewire is attached to a means for providing rotation and/or vibration of the guidewire tip which extends beyond the tip of the catheter. Rotational and/or vibrational movement of the guidewire tip assists in crossing calcified or otherwise difficult lesions. In some embodiments, the tip of the guidewire is configured to facilitate cutting through the lesion, for example by having a pointed, angled, star-shaped, or auger-like tip. In some embodiments, the guidewire is equipped to provide both RF and rotational/vibrational assistance in crossing the lesion.

The combined device provides several advantages over the prior art. First, it provides a safer method to cross the occlusion by providing visualization of the intravascular lumen so that the physician can avoid damaging the vessel wall. Second, visualization of the area in front of the device can be used to guide the recanalizing device through the occlusion, which often involves navigating a tortuous path. Third, by combining the visualization catheter with the guidewire that is used to initially traverse the patient's vasculature, it is not necessary to withdraw the guidewire and replace it with a different therapeutic device, thereby simplifying the procedure and reducing associated risks to the patient. As a result, the device of the present invention can safely and effectively open the occluded blood vessel for renewed blood flow, or to facilitate the use of other interventional technique such as angioplasty or stent placement.

According to an aspect of the present invention, an elastic or superelastic material is utilized as a structural material for the new micromanipulator. Elasticity or superelasticity is therefore an important design parameter for compliant mechanisms of the micromanipulator. In principle, when a compliant mechanism is deformed with an actuator, strain energy is stored inside the underlying structure during deformation (elastic and plastic). The stored energy is then directly utilized to produce a bias force to return the structure to its original shape.

In some embodiments, Shape Memory Alloys (SMAs) are implemented as main actuators for the micromanipulator. The compliant mechanism is actuated with SMA contraction as well as rotation motion to maximize output displacement. By activating the SMAs, it is possible to achieve approximately ±30° angular deflections. It is anticipated that the compliant mechanism can be designed to accommodate two other SMAs in an orthogonal direction, in which case, the compliant mechanism can be manipulated with two degree-of-freedom, which would provide the micromanipulator with full 3-D scanning motions.

According to an aspect of the invention, a Nd:YAG laser is implemented in the fabrication of the compliant structure out of a tube. A tubular nitinol structure with compliant mechanism can successfully fabricated using laser machining with a laser beam size of about 30 μm. The outer diameter of the tube is advantageously about 800 μm and the wall thickness is about 75 μm. Preferably, the actual feature size is about 25 μm, which is mostly limited by the size of the laser beam. Thus, by reducing the beam size, resolution of the laser machining can be enhanced.

Micromanipulators of the present invention with novel features such as structural compliance, elasticity/superelasticity, tubular structure, etc. are particularly useful in the fields of intravascular ultrasound (IVUS) imaging and intravascular intervention.

A preferred embodiment of the invention is a medical device to cross a vascular occlusion comprising an intravascular catheter having a proximal end and distal end, and at least one lumen traversing along the longitudinal axis of the catheter; a guidewire disposed in the at least one lumen, the guidewire having a proximal end and a distal end, where the guidewire can be moved axially within the lumen such that the distal end of the guidewire can be extended beyond the distal end of the catheter; an ultrasound apparatus having an ultrasound transducer, the ultrasound apparatus located at the distal end of the catheter, where the ultrasound apparatus is configured to provide real-time imaging of an area proximate to a distal tip of the catheter; where the device is configured to provide energy to a distal end of the device, where the energy is selected from the group comprising radio-frequency energy, rotary energy, vibrational energy, or oscillatory energy; and where the energy is sufficient to assist the device in crossing a vascular occlusion.

In any of the embodiments, the medical device can further comprise a radio frequency source generator attached to the proximal end of the guidewire, where the guidewire is configured to emit radio frequency energy from only a distal tip of the distal end of the guidewire. In any of the embodiments, the device can have a bi-polar configuration, and the catheter can further comprise an electrode on the distal end of the catheter.

In any of the embodiments, the ultrasound device can be configured to provide real-time imaging of at least an area in front of the distal tip of the catheter. In any of the embodiments, the ultrasound device can be configured to provide real-time imaging of at least an area at a right angle to the longitudinal axis of the catheter at the distal tip of the catheter.

In any of the embodiments, the radio-frequency energy can have a frequency of about 200 kHz and about 700 kHz.

In any of the embodiments, the distal tip of the guidewire can have a cutting means to facilitate crossing a vascular lesion.

In any of the embodiments, the medical device can further comprise a motor attached to the proximal end of the guidewire, where the motor is configured to rotate the guidewire around its longitudinal axis, to vibrate a distal tip of the guidewire, or to oscillate a distal tip of the guidewire. In any of the embodiments, the motor can rotate the guidewire around its longitudinal axis at a rate of about 1000 rpm to about 9000 rpm. In any of the embodiments, the medical device can further comprise a radio frequency source generator attached to the proximal end of the guidewire, where the guidewire is configured to emit radio frequency energy from only a distal tip of the distal end of the guidewire.

In any of the embodiments, the medical device can further comprise an optical fiber disposed in an additional lumen, the optical fiber having a proximal end and a distal end, where the optical fiber is configured to perform optical coherence tomography.

In any of the embodiments, the medical device can further comprise a second lumen traversing along the longitudinal axis of the catheter, where the second lumen is in communication with the environment at the distal end of the catheter, such that suction applied to the proximal end of the second lumen can be used to aspirate the environment proximate to the distal end of the catheter.

In any of the embodiments, the medical device can further comprise a radio frequency source generator attached to the proximal end of the catheter, where the catheter is configured to emit radio frequency energy from only an electrode on the distal tip of the catheter. In any of the embodiments, the device can have a bi-polar configuration, where the catheter can further comprise a second electrode on the distal end of the catheter. In any of the embodiments, the medical device can further comprise a motor attached to the proximal end of the guidewire, where the motor is configured to rotate the guidewire around its longitudinal axis, to vibrate a distal tip of the guidewire, or to oscillate a distal tip of the guidewire.

In any of the embodiments, the ultrasound apparatus can comprise a compliant apparatus sized for intravascular use having no mechanical joints and capable of being manipulated through at least one degree of freedom without permanent deformation, the compliant apparatus comprising: a tubular structure having an axis and formed from a tube made of a material having a reversible structural behavior; at least one compliant mechanism integrally formed from the tube by removing material from the tube to facilitate bending motion; and at least one force-generating actuator attached to the compliant apparatus for manipulating the compliant apparatus by bending the at least one compliant mechanism away from the axis; where the ultrasound transducer is coupled to the compliant apparatus.

Another embodiment of the invention is a method for crossing a vascular occlusion comprising: inserting the distal end of a guidewire having a distal end and a proximal end into the vasculature of an animal having a vascular occlusion; advancing the distal end of the guidewire through the vascular to the occlusion; passing the distal end of an intravascular catheter having a proximal end, a distal end, and at least one lumen traversing along the longitudinal axis of the catheter over the proximal end of the guidewire such that the proximal end of the guidewire is disposed in the lumen; advancing the distal end of the catheter over the guidewire until the distal end of the guidewire is proximate to the occlusion; using an ultrasound apparatus having an ultrasound transducer, the ultrasound apparatus located at the distal end of the catheter, to provide real-time imaging of the environment proximate to a distal tip of the catheter; advancing the distal tip of the guidewire through the occlusion under the guidance of the real-time imaging until the guidewire encounters a portion of the occlusion which cannot be crossed; and providing energy to the distal end of the guidewire where the energy is at least radio-frequency, rotational, vibrational, or oscillatory, such that the energy permits the advancement of the distal tip of the guidewire through the occlusion.

In any of the embodiments, the energy can be radio-frequency energy and the energy can be provided by activating a radio-frequency generator attached to the proximal end of the guidewire such that radio-frequency energy is emitted from only a distal tip of the guidewire; and the radio-frequency energy can have a frequency of about 200 kHz and about 700 kHz.

In any of the embodiments, the energy can be rotational, vibrational or oscillatory energy and the energy can be provided by activating a motor attached to the proximal end of the guidewire such that a distal tip of the guidewire is rotated, vibrated, or oscillated.

In any of the embodiments, the motor can rotate the guidewire around its longitudinal axis at a rate of about 1000 rpm to about 9000 rpm.

In any of the embodiments, the energy can be radio-frequency energy and the energy can be provided by activating a radio-frequency generator attached to the proximal end of the catheter such that radio-frequency energy is emitted from an electrode on the distal tip of the catheter; and the radio-frequency energy can have a frequency of about 200 kHz and about 700 kHz.

In any of the embodiments, the ultrasound device can be configured to provide real-time imaging of at least the area in front of the distal tip of the catheter. In any of the embodiments, the ultrasound device can be configured to provide real-time imaging of at least an area at a right angle to the longitudinal axis of the catheter at the distal tip of the catheter.

In any of the embodiments, the intravascular catheter can further comprise an optical fiber disposed in a second lumen, the optical fiber having a proximal end and a distal end, where the optical fiber is configured to perform optical coherence tomography; and the embodiment can include use of the optical fiber to perform optical coherence tomography to provide real-time imaging of the environment proximate to a distal tip of the catheter.

Still further objects and advantages of the present invention will become apparent to one of ordinary skill in the art upon reading and understanding the drawings and detailed description of the preferred embodiments disclosed herein. As it will be appreciated by one of ordinary skill in the art, various changes, substitutions, and alternations can be made without departing from the principles and the scope of the present invention. As such, the drawings disclosed herein are for purposes of illustrating embodiments of the present invention and are not to be construed as limiting the present invention.

### BRIEF DESCRIPTION OF THE DRAWINGS

FIG. 1 compares the second moment of inertia between a plate form and a tubular structure.

FIGS. 2A-2B show two tubular structures each with a built-in compliant mechanism in different design configuration.

FIG. 3 schematically shows an ultrasound transducer coupled to a micromanipulator having the compliant structure of FIG. 2A and two SMA actuators configured to actuate the compliant mechanism thereof.

FIG. 4 is a photograph showing an exemplary compliant structure of FIG. 2A having no mechanical joints and made of a nitinol tube with a built-in compliant mechanism.

FIGS. 5A-5B are photographs showing a micromanipulator having the compliant structure of FIG. 4 and two SMA actuators configured to actuate the compliant mechanism thereof.

FIG. 6 schematically shows an implementation of FIG. 2A useful for a catheter steering system. The tubular compliant structure has multiple segments of compliant mechanisms each individually controllable via SMA actuators assembled therewith.

FIG. 7 schematically shows an implementation of FIG. 6 coupled with an ultrasound transducer.

FIG. 8 schematically shows an exemplary intravascular imaging device embodying the implementation of FIG. 7, the imaging device integrated with a cooling system.

FIG. 9 is a photograph showing another exemplary compliant structure under loading in a bulging-out configuration.

FIG. 10 schematically shows a tubular structure with a built-in compliant mechanism that enables the bulging-out configuration of FIG. 9.

FIG. 11A is a side view showing an embodiment of a medical device where the distal tip of the guidewire is equipped to emit radio-frequency energy. FIG. 11B is a perspective view of the distal end of the device.

FIG. 12A is a side view showing an embodiment of a medical device where the distal tip of the guidewire is equipped to rotate, vibrate, or oscillate. FIG. 12B is a perspective view of the distal end of the device.

FIGS. 13A-D are side views showing embodiments of the distal tip of the guidewire.

FIG. 14A is a side view showing an embodiment of a medical device comprising an optical fiber for optical coherence tomography. FIG. 14B is a perspective view of the distal end of the device.

FIG. 15 is a side view showing an embodiment of the distal end of a medical device having lumens with openings in the distal end of the device.

FIG. 16 is a side view showing an embodiment of a medical device where the distal end of the catheter is equipped to emit radio-frequency energy.

FIGS. 17 is a schematic diagram showing an embodiment of a method for crossing a vascular occlusion.

### DETAILED DESCRIPTION OF THE PREFERRED EMBODIMENT

To address the fracture toughness and stress issue, a superelastic material such as nitinol is utilized as a structural material for the micromanipulator of the present invention. Thus, superelasticity is implemented as an important design parameter for compliant mechanisms disclosed herein. In principle, when a compliant mechanism is deformed with an actuator, strain energy is stored inside the underlying structure during deformation (elastic and plastic). The stored energy is then directly utilized to produce a bias force to return the structure back to its original shape. However, an elastic material such as stainless steel can also be utilized as a structural material for compliant mechanisms if the fracture and stress issue can be appropriately addressed with elasticity as a design parameter.

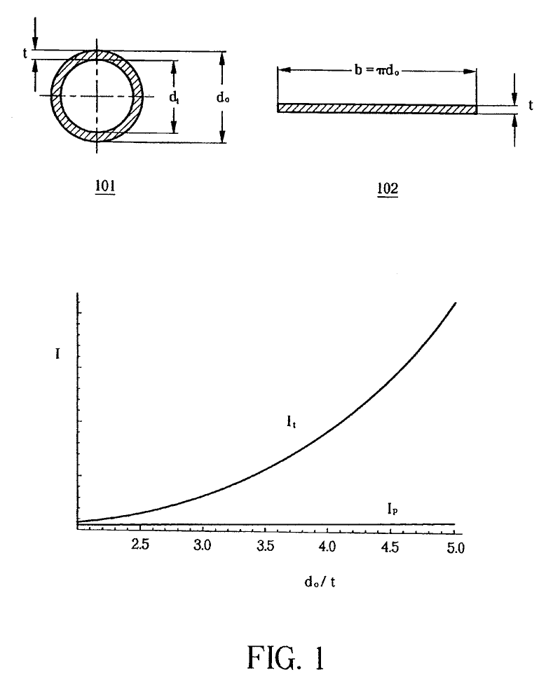

To shape a nitinol structure, there are two fabrication processes currently commercially available: chemical etching and laser machining. However, these two processes are not able to precisely control etching depth. T thus, it is very difficult to have a variation in thickness and, consequently, the thickness of the mechanism determines the substrate thickness. This presents another issue in design, which is structural rigidity. For instance, if the substrate thickness is on the order of tens of microns, the supporting structure also starts deflecting as the mechanism moves. This deflection at the supporting structure, which is supposed to be fixed, directly contributes to loss of output displacement. Structural rigidity is mostly a shape factor, which is related to flexural modulus, EI. Considering the structural rigidity, a tube shape 101 is more attractive than a plate form 102 as demonstrated in FIG. 1, where

$$\begin{matrix}{I}_{p} = \frac{b {t}^{3}}{12} = \frac{π {d}_{o} {t}^{3}}{12} & ( 1 ) \\ {I}_{t} = \frac{π ( {d}_{o}^{4} - {d}_{i}^{4} )}{64} = \frac{π ( {d}_{o}^{4} - {( {d}_{o} - 2 t )}^{4} )}{64} & ( 2 )\end{matrix}$$

Iₜ and Iₚ respectively represents the second moment of inertia of a tube and a plate. The lengths of the plate and the tube are assumed to be the same for correct comparisons in equations (1) and (2).

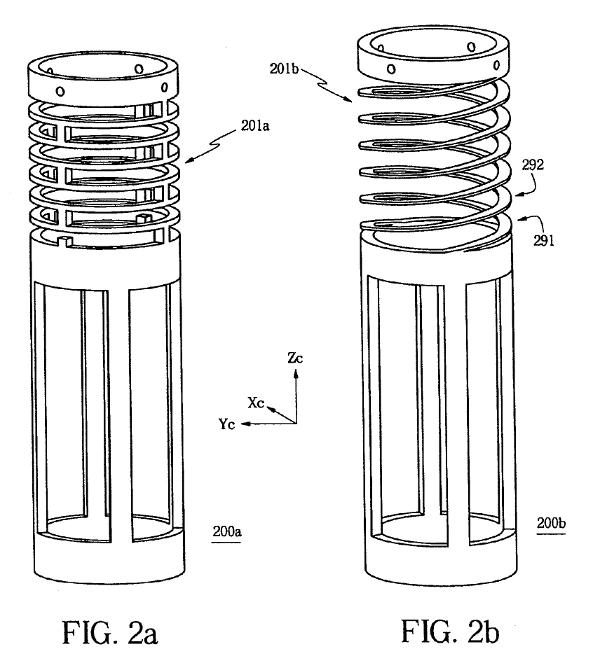

FIG. 1 shows that there is an exponential difference in structural rigidity as dₒ/t increases, which is a reasonable estimation for the compliant mechanism. Thus, the tube was selected as a basic form of structure for the compliant mechanisms. FIG. 2A illustrates an exemplary tubular structure 200a with a built-in compliant mechanism 201a. FIG. 2B illustrates another exemplary tubular structure 200b with a built-in compliant mechanism 201b in a helical configuration having helix 291 and helix 292 intertwined in a “double helix”-like fashion. The mechanism design can be any shape and/or configuration as long as it utilizes structural compliance (elasticity and/or superelasticity) as a main design parameter. Similarly, as one skilled in the art would appreciate, the rest of the tubular structure can be in any suitable configuration, size, and length, etc., optimized for a particular application and thus is not limited to what is shown here. Moreover, in addition to nitinol, other flexible, resilient biocompatible metal or polymer materials can also be utilized as long as they have reversible structural behaviors, i.e., have elastic and/or superelastic behaviors while actuated.

As illustrated in FIG. 2B, compliant mechanisms can be in a “double helix” configuration. It is desirable with the present invention that any bending strain of the compliant mechanisms is distributed substantially evenly along their entire lengths. This reduces peak strain, which in various embodiments, can be, 4% or less, 3% or less, 2% or less and 1% or less. The “double helix” configuration provides greater symmetry in motion and provides a more even bending It is desired that the stiffness of compliant mechanisms in different directions be substantially the same.

In various embodiments, the elastic bending strength of the compliant mechanisms is customized in order to match with that of the actuators. In some embodiments, the actuators have slightly stiffer elastic bending strengths than those of the compliant mechanisms. In one embodiment, the compliant mechanisms are stiffer than the actuators when the actuators are relaxed, and the compliant mechanisms are softer than the actuators when the actuators are active. It is desirable to provide compliant mechanisms in configurations, such as those of the “double helix” configurations, that have as little stress concentration as possible.

According to the present invention, the strain of a compliant mechanism is distributed, while minimizing the occurrence of strain location. The mechanical characterization of a compliant mechanism can be tuned by modifications in, (i) stiffness, (ii) peak strain (maximum strain), (iii) size, (iv) fatigue life, and the like. In one embodiment, the upper limit of strain is no more than 4%. The bending stiffness depends on actual application. By way of illustration, and without limitation, the bending stiffness of a compliant mechanism can be at least 0.5 N-mm and no more than 10 N-mm. In various embodiments, compliant mechanisms are stiffer than the imaging device. The associated actuators are also stiffer than the imaging device. The actuators need a longer thermal time constant than the imagining device.

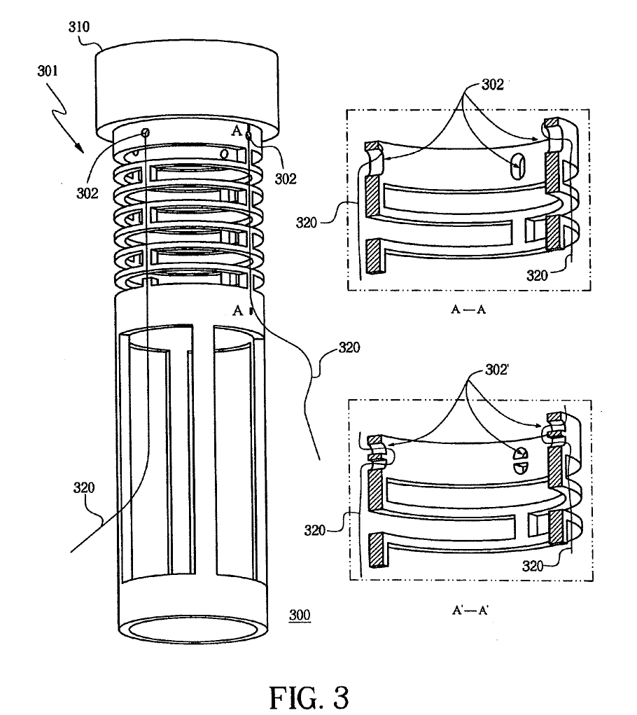

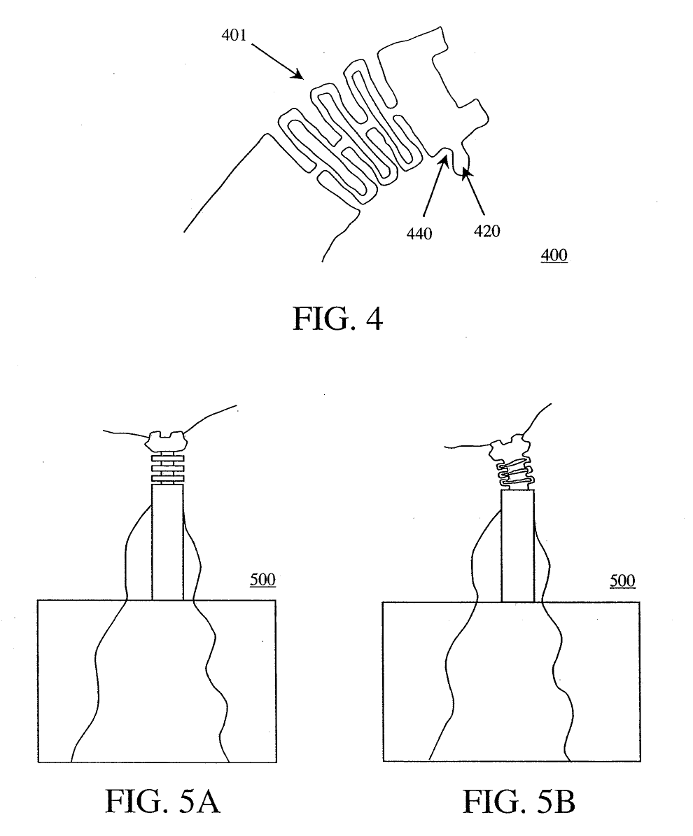

FIG. 3 schematically shows, according to an aspect of the invention, a micromanipulator 300 tightly coupled with an ultrasound transducer 310 for image scanning. Micromanipulator 300, as well as the other embodiments of micromanipulators disclosed herein, provide for steering, viewing and treatment at sites within vessels of the body, as well as for industrial applications. As discussed before, most of the research efforts on ultrasonic imaging system for intravascular intervention utilized ultrasonic transducers to inspect sidewall images inside blood vessels. These transducers are turned inside at high speed to capture the inner images, which do not provide any information about the front images. As one skilled in the art would appreciate, it would be extremely helpful if cardiologists can see the cross-section (front images) of the blood vessels in front of the device used to remove the stenosis. To catch the front images in various angles needed to create the images in front of the device, a micromanipulator is required to maneuver the transducer and generate a scanning motion.

The micromanipulator 300 enables the ultrasound transducer 310 to be directly coupled to the compliant mechanisms 301. In this fashion, the rotational center of the transducer 310 for the scanning motion is substantially closer to the rotational axis of the mechanisms 301. This novel configuration can produce images with much better resolutions than known devices. In an embodiment, SMAs (Shape Memory Alloys) are implemented as main actuators 320 for the micromanipulator 300. To allow the SMAs 320 be attached thereto, the micromanipulator 300 can have one or more attachment points or built-in micro structures such as welding-enabling structures 302 as shown in a cross-sectional view A-A and clamping-enabling structures 302′ as shown in another cross-sectional view A′-A′. In some embodiments, the SMAs 320 are attached to the compliant apparatus via the one or more attachment points or welding-enabling structure 302 using a laser having a laser beam size of about 200 μm or less. In some embodiments, the SMAs 320 are fastened to the compliant apparatus via the built-in clamping-enabling structures 302′.

The compliant mechanisms 301 are actuated with SMA 320 actuators based on shape memory effects including contraction as well as rotation motion to maximize output displacement. As one skilled in the art can appreciate, the SMA actuators can be in any shape such as wire, spring, coil, etc. and thus is not limited to what is shown here.

The amount of continuous power applied to all of the actuators is 1 W or less, with a peak power of 10 W or less. It will be appreciated that the micromanipulator of the present invention can have at least two actuator. Additional actuators can be utilized, subject to the ability to manufacture, cost, size, and like.

According to an aspect of the invention, a Nd:YAG laser was implemented in fabricating compliant structures out of nitinol tubes. The laser has a wavelength of 1.06 μm and an average power of 75 W. The cutting depth of the laser is about 125 μm. Nd:YAG lasers as well as other lasers suitable for the laser machining are known in the art and thus are not further described herein. Referring to FIG. 4, a compliant structure 400 was successfully fabricated out of a nitinol tube using laser machining. The outer diameter of the nitinol tube is about 800 μm and the wall thickness is about 75 μm. The compliant structure 400 can be characterized as a tubular nitinol structure with a built-in compliant mechanism 401 and loading points 440. The compliant structure 400 shown in FIG. 4 is actuated with a SMA actuator 420 via one of the loading points 440. It is also useful to pattern the compliant structures with holding structures (not shown) for temporarily holding the SMA actuator during assembly and to decrease stress upon the SMA actuator at the attachment point in the final device. In this embodiment, the compliant structure 400 has features about 30 μm in size. In practice, actual feature size is mostly limited by the size of the laser beam, which was about 25 μm in this example. It will be apparent to one skilled in the art that, by reducing the beam size, the resolution of the laser machining can be enhanced.

The size of the various elements of micromanipulators of the present invention can be customized depending on applications. For example, if it is desired to insert a micromanipulator into the inner diameter of another device, the diameter of the micromanipulator is selected so that the micromanipulator can fit in the inner diameter of that device. In a more specific example, for a coronary artery, it is desired to have a micromanipulator with a diameter of 2 mm or less. For larger vessels, the diameter of micromanipulator can be 4 mm or less.

The tubular nitinol compliant structure 400 was tested under cyclic loading. Specifically, SMA actuators generated a cyclic motion of the compliant structure 400 at 10 Hz under water. The compliant structure 400 successfully endured the mechanical loading test while it was actuated. No mechanical failure was noticed up to 20,000 cycles.

FIGS. 5A-5B show a micromanipulator 500 having a compliant structure as shown in FIG. 4 and two main actuators in the form of SMA wires successfully assembled therewith. In various embodiments, the actuators of micromanipulator 500 provide angular deflection of at least ±20°. In the embodiments illustrated in FIGS. 5A-5B the actuators are activated, resulting in ±40° angular deflections. The micromanipulator 500 can be assembled with two other actuators in an orthogonal direction. The micromanipulator 500 so assembled will be able to manipulate the compliant mechanism with two degree-of-freedom, which would provide full 3-D scanning motions. 3-D scanning motions can be achieved by utilizing an actuator for one direction of deflection, and then a second actuator for the second direction of deflection. It will be appreciated that the second direction of deflection can be achieved by rotation movement, for example by way of illustration, and without limitation, in a helical type of scan.

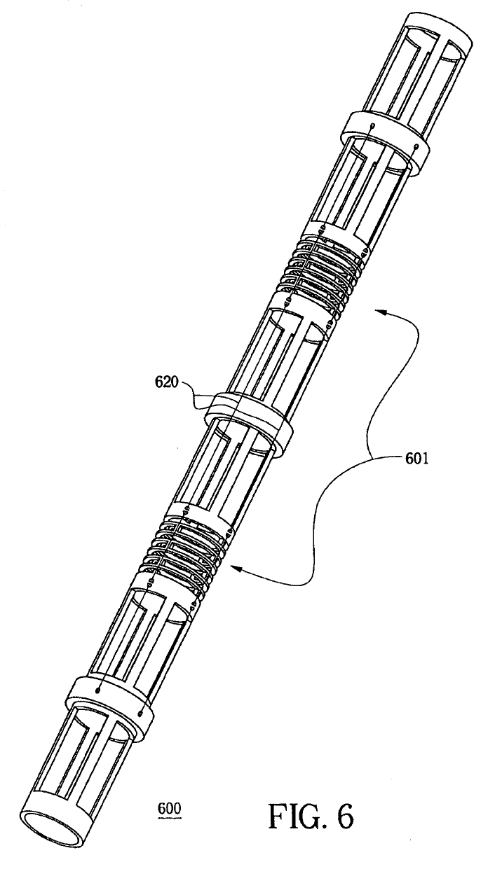

In addition to being particularly useful in ultrasound intravascular interventional devices, systems, and applications, the present invention can also be useful in catheter steering related applications including but not limited to any vessels in the body, such as those in neurology, biliary vessels, the fallopian tubes, coronary vessels (including peripheral vessels), and the like. It will be appreciated that the present invention can also be utilized for industrial applications as mentioned above. In a conventional catheter steering system, it is difficult to steer a small catheter inside human blood vessels, especially in small artery. However, by implementing a compliant structure with multiple segments of compliant mechanisms in various configurations and individually controlling each segment, it is possible to generate intricate motions and steer the catheter in any direction, even in a tiny area. For example, a catheter steering system implementing a micromanipulator 600 according to the present invention may include multiple segments of compliant mechanisms 601 actuated with SMAs 620, as shown in FIG. 6. These tubular compliant mechanisms are arranged in various configurations for intricate motions of the micromanipulator. Such catheter steering system is particularly useful for intravascular applications including imaging and therapy.

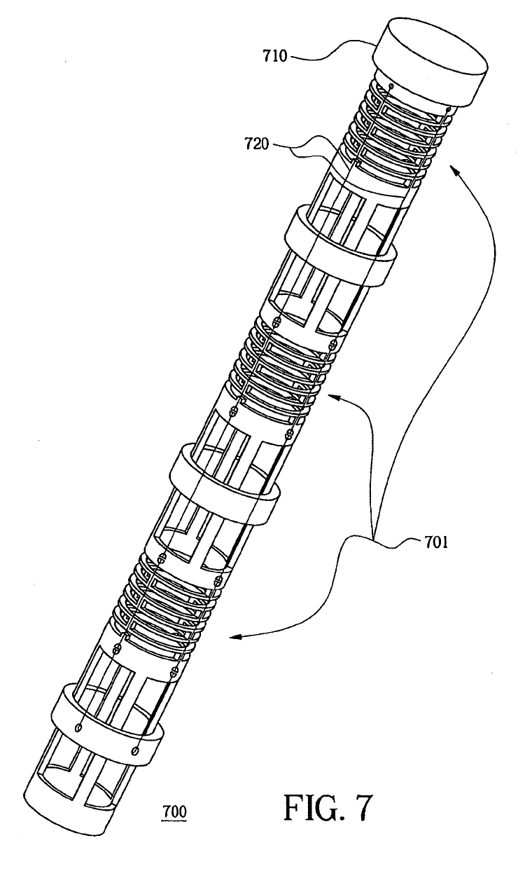

FIG. 7 shows a micromanipulator 700 with an ultrasound transducer 710 directly coupled thereto at one end of the micromanipulator 700 for forward imaging. The micromanipulator 700 has multiple segments of compliant mechanisms 701 actuated with SMAs 720. Multiple segments of compliant mechanisms 701 are useful for vessels with different curvatures. For example, one section of a vessel may require a larger curvature than another area. Therefore, multiple segments of compliant mechanisms 701 make it easier to traverse through a vessel with different curvatures. A user of the system controls individual segment's compliant mechanism via a user interface of an external electronic circuitry, e.g., a computer (not shown).

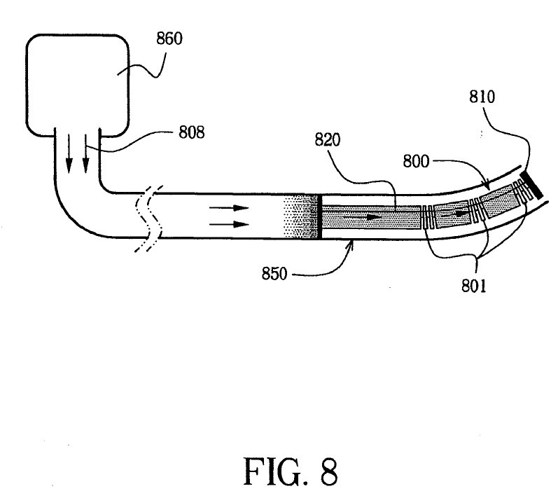

When SMAs are implemented as main actuators for the micromanipulator, the performance (e.g., bandwidth and endurance) of the manipulator and devices associated therewith, e.g., an imaging or therapeutic device, can be substantially enhanced by regulating the temperature of the SMAs. Regulation of the temperature can be controlled by any suitable cooling system (e.g., peristaltic pump and IV pump). FIG. 8 shows a micromanipulator 800 having multiple segments of compliant mechanisms 801 actuated by SMAs 820. The micromanipulator 800 is coupled to an ultrasound transducer 810 and steered by SMA actuators 820. A plastic tube, catheter 850, encapsulates the micromanipulator 800, SMAs 820, transducer 810, etc. A cooling system 860, comprising a pumping means and cooling fluid, provides a constant fluid flow 808 to the micromanipulator 800 to prevent the SMAs 820 from overheating during normal operation. Here, the cooling fluid can be any biocompatible solution such as water or saline.

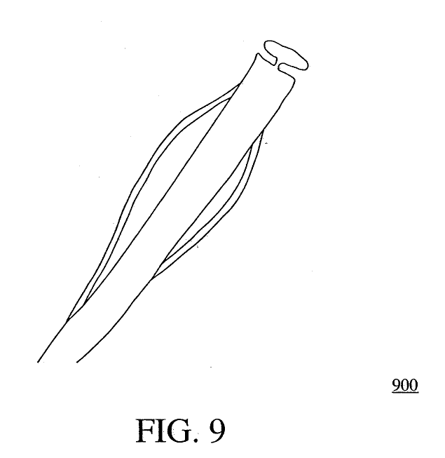

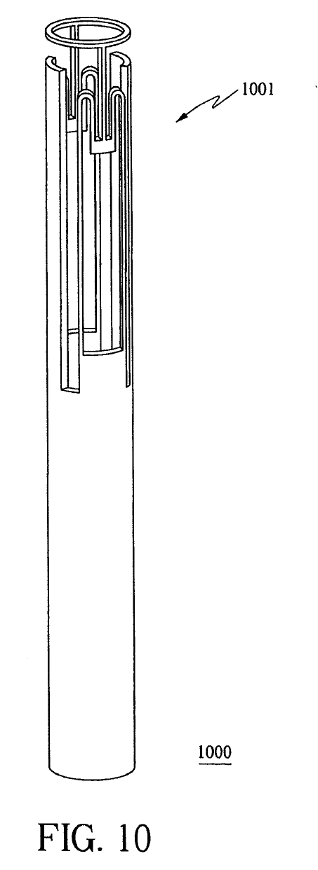

Another application includes utilizing the novel design disclosed herein for angioplasty. Currently, depending on the size of arteries that need to be cleared, surgeons use different sizes of balloons during operation. This means that they would have to change balloon sizes several times and each balloon must be taken out of the body for another balloon to be inserted in. As one skilled in the art would appreciate, the exchange of balloons is a necessary but undesirable procedure. Implementing the compliant mechanisms disclosed herein, it is possible to cover certain ranges of balloon sizes with one single device, as exemplified in FIG. 9. FIG. 9 shows an actual compliant structure 900 under loading in a bulging-out configuration. FIG. 10 schematically shows a tubular structure 1000 with a built-in compliant mechanism 1001 that enables the bulging-out configuration of FIG. 9. It will be apparent to one skilled in the art that the compliant mechanisms disclosed herein have more capabilities in terms of pressure and deployment control than prior art surgical balloons. Moreover, with the present invention, the need to exchange balloons during operation can be substantially reduced or eliminated, thereby simplifying and possibly shorting the angioplasty procedure, making it easier on the surgeons and safer for the patients. The advantages of the present invention are innumerable.

To date, we are not aware of any methods for manufacturing compliant mechanisms out of a nitinol tube for intravascular intervention. Similarly, we are not aware of anyone implementing laser machining as a main fabrication tool for constructing compliant mechanisms. The present invention advantageously utilizes structural compliance, elasticity/superelasticity, and strain energy as a restoring force. Compliant structures and micromanipulators based on these features (structural compliance, elasticity/superelasticity, tubular structure, etc.) as disclosed herein are believed to be unprecedented. The present invention is useful in many fields, e.g., a micromanipulator implemented with an ultrasound transducer such as one shown in FIG. 3 would be useful in intravascular ultrasound (IVUS) applications and particularly in forward imaging systems. A micromanipulator implemented with multiple segments of compliant mechanisms would be useful in steering a catheter in any direction, even in a tiny area. In various embodiments, the present invention can utilize a variety of different interventions for treatment, including but not limited to laser, rotor blader, RF, mechanical, stiff guide wire, microwave, ultrasound, chemical, and the like.

A micromanipulator implemented with a bulging-out configuration as shown in FIGS. 9-10 would be useful in angioplasty and other types of operations where exchanging different sizes of balloons and the like is necessary but undesirable. The micromanipulator of the present invention is made of a monolithic material, e.g., a nitinol tube with a reversible structural behavior, with a built-in compliant mechanism. Since there are no mechanical joints, the micromanipulator can be very small and can facilitate surgical operations in a minimally invasive fashion.

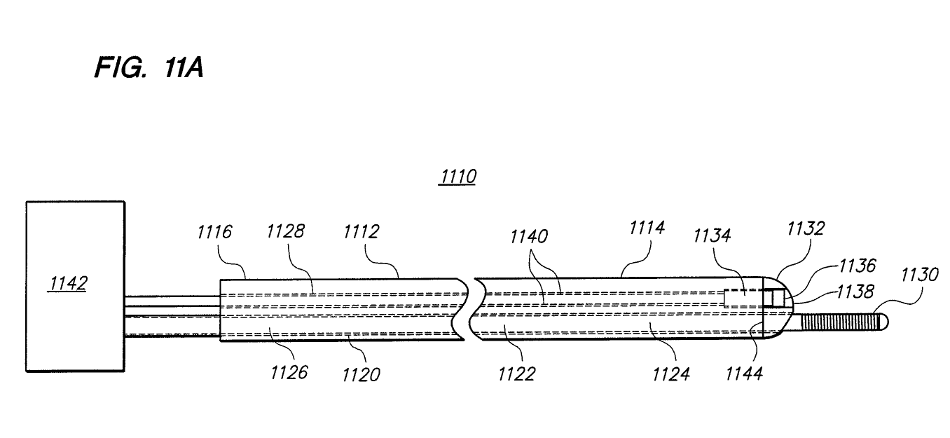

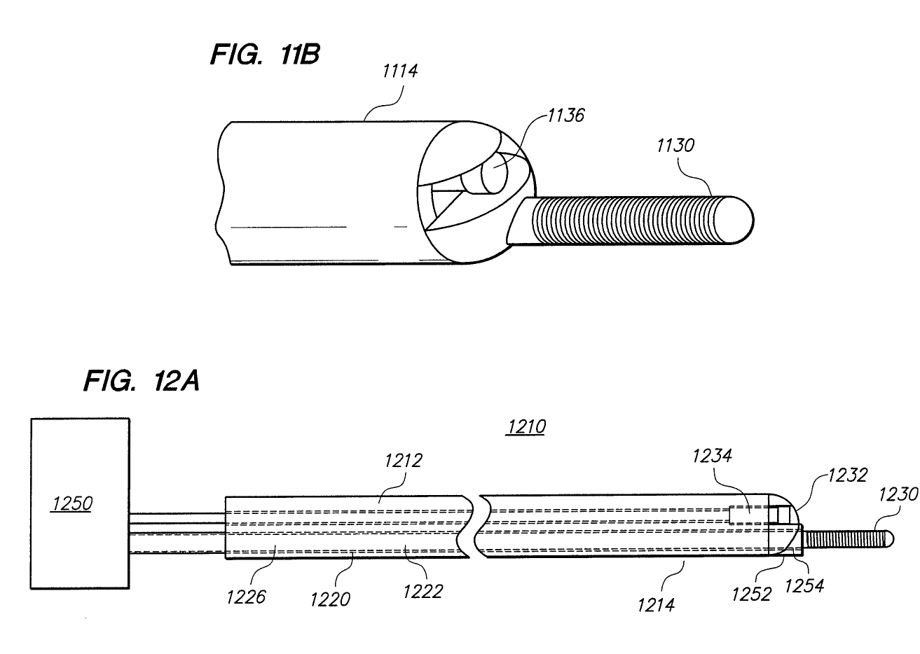

FIG. 11 is another embodiment of the disclosed invention. In FIG. 11 A, a medical device 1110 comprising an elongate member such as an intravascular catheter 1112 is shown with a distal end 1114 and proximal end 1116. Intravascular catheters are well known in the art, and the common features of such catheters are not shown. These include operative elements at the proximal end to allow the operator of the catheter to manipulate the catheter while in the vasculature of the patient, as well as ports attached to the proximal end to allow for the insertion of various implements, fluids, etc. into one or more lumens or channels in the catheter. FIG. 11 illustrates a catheter with at least one lumen 1120 traversing the longitudinal axis of the catheter. Disposed within the lumen is an occlusion crossing means, e.g. a guidewire 1122 which has a distal 1124 and proximal 1126 end. The guidewire is disposed within the lumen 1120 of the catheter such that the guidewire can move rotationally and/or longitudinally within the lumen 1120. The distal end of the lumen 1120 is open, such that the distal tip 1130 of the guidewire 1122 can be extend past the distal tip 1132 of the catheter 1112. The lumen(s) can optionally have one or more openings on one or more sides of the catheter.

An intravascular ultrasound (IVUS) imaging device 1134 is located at the distal end 1114 of the catheter, preferably, all though not necessarily, at the distal tip 1132 of the catheter 1112. The ultrasonic imaging device has an ultrasound transducer 1136 located on its distal tip. The transducer is connected by one or more wires 1140 to the other components of an ultrasound imaging system, such as an ultrasound source generator, receiver and computer (not shown) located near the proximal end 1116 of the catheter 1112. In some embodiments, the ultrasonic imaging device is connected wirelessly to one or more components of the ultrasound imaging system.

Preferably, the ultrasound imaging device 1134 is configured to provide real-time imaging of the environment in front of the distal tip 1132 of the catheter, i.e. forward-looking. In other configurations, the ultrasound device can be configured to provide a view of the vessel wall beside the distal end 1114 of the catheter (i.e., side-looking). Although the ultrasound imaging device 1134 is shown located on the distal tip 1132 of the catheter oriented generally along the longitudinal axis of the catheter, the ultrasound imaging device 1134 can be located on the side of the distal end 1134 or distal tip 1132 of the catheter, oriented generally at a right angle to the longitudinal axis of the catheter, or at an angle oblique to the longitudinal axis. The device can be oriented at any angle from the longitudinal axis, depending on the field of view desired, wherein the angle is about 0, 10, 20, 30, 40, 50, 60, 70, 80, or 90 degrees. In other configurations, the device is able to provide a wide field of view, such that it provides both side and forward-looking capabilities. The device can provide a field of view that is about 5, 10, 15, 20, 30, 40, 50, 60, 70, 80, 90, 100, 110, 120, 130, 140, 150, 160, 170, 180 degrees, or more, or any range defined by any two of these values. The guidewire 1122 can be used to safely and effectively cross an occlusion of a blood vessel under the visual guidance provided by the ultrasound imaging device 1134. In a preferred embodiment, the IVUS imaging device 1134 comprises the compliant mechanism disclosed herein, for example, as disclosed in FIG. 3, although the use of other ultrasound devices is contemplated.

In some embodiments, the catheter 1112 has one or more additional lumens traversing the longitudinal axis of the catheter 1112. For example, the wire(s) 1140 shown in FIG. 1 can be disposed inside a lumen 1128 traversing the longitudinal axis of the catheter in the location illustrated by the wire(s) 1140. This or another additional lumen (not shown) can be used to supply a fluid to the IVUS imaging device 1134. The fluid can be used for cooling one or more portions of the ultrasound device (e.g. the transducer 1134 or an actuating means). Optionally, the distal tip 1132 of the catheter can be enclosed with an ultrasound transparent cover 1138. In such an embodiment, the fluid provided through the additional lumen(s) can also be used to flush the area around the IVUS imaging device 1134 to ensure that the area is free of debris or bubbles which would interfere with the performance of the ultrasound device. In embodiments where the distal tip of the catheter is capped, it is desirable to provide a means for the fluid to circulate through the area around the ultrasound transducer, such as a lumen (not shown) to return the fluid to the proximal end of the catheter or an opening in the distal portion of the catheter so that the fluid can escape. The fluid is preferably biocompatible.

In some embodiments of the invention, the guidewire 1122 is connected to a radio frequency (RF) generator 1142. The guidewire is configured so that it emits radio frequency energy from its distal tip 1130. This can be achieved by using a guidewire with electrical insulation along its entire length, with only the portion of the distal tip 1130 of the guidewire which is used to emit RF energy exposed. Preferably, less than about 1 mm of the distal tip of the guidewire is exposed. However, also contemplated is exposing an amount that is, is about, is at least, is at least about, is not more than, or is not more than about, 2.0, 1.75, 1.5, 1.25, 1.0, 0.75, 0.5, 0.25, or 0.1 mm, or falls within a range defined by any two of these values. All sides and the end of the distal tip 1130 can be exposed, or just the end and/or one or more sides of the distal tip 1130 can be exposed. If the distal tip 1130 is generally circular, the entire circumference of the distal tip 1130, or only a portion can be exposed.

In some embodiments, the RF tip is in a mono-polar configuration where the patient's body is electrically grounded through a ground patch (not shown). In other embodiments, the RF tip is in a bi-polar configuration where the ground wire 1144 is located on the distal end 1114 of the catheter. The ground wire 1144 can be connected to the RF generator 1142 by a wire (not shown) disposed in the lumen 1120 or the channel provided for the ultrasound wire 1140. In some configurations the polarity of the RF wires is reversed, such that the wire 1144 on the catheter is the RF emitting wire and the distal tip 1130 of the guidewire is the ground wire.

Traditional guidewires have difficulty crossing hardened or calcified lesions. Using the power of the RF tip 1130 in either the mono-polar or bi-polar configuration, the guidewire 1122 can cross the occlusions easily and effectively. The preferred frequency of the RF energy is between about 200 kHz and about 1 MHz. In some embodiments, the minimum frequency is, is about, is at least about, is no more than, or is no more than about 200, 250, 300, 350, 400, 450, 500, 550, 600, 650, 700, 750, 800, 850, 900, 950, or 1000 kHz, or falls within a range defined by any two of these values. Because the RF enabled guidewire can pose a greater danger of damage to the blood vessel, it is preferable to use the RF guidewire under the real-time visual guidance provided by the ultrasound device 1134 because it is safer and more effective.

FIG. 11B is a perspective view of the distal end 1114 of the medical device shown in FIG. 11A. The ultrasound transducer 1136 and distal tip 1130 of the guidewire are shown.

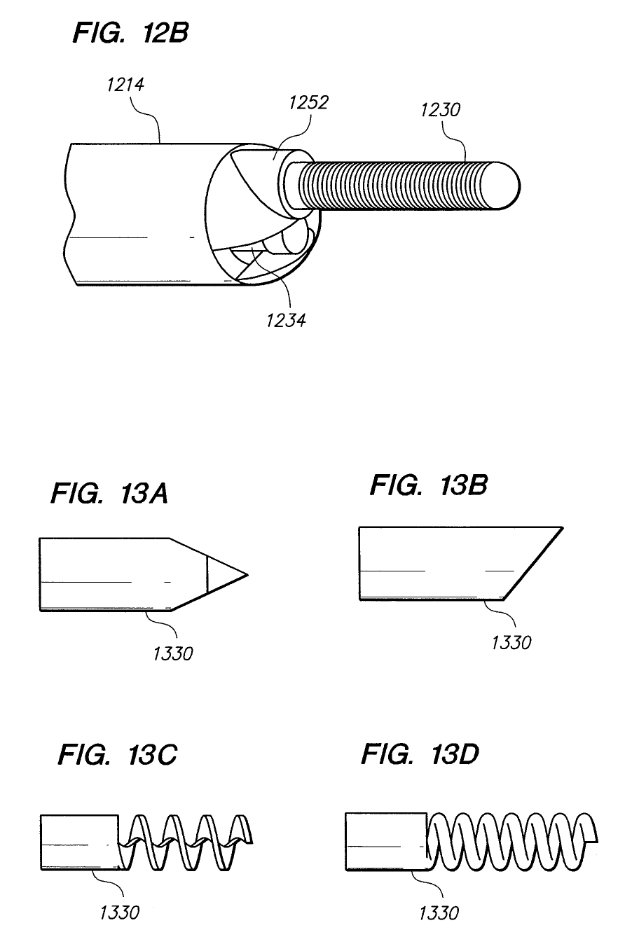

FIG. 12 illustrates another embodiment of the invention similar to the one illustrated in FIG. 11. In FIG. 12A, a medical device 1210 is depicted in which a guidewire 1222 located in a lumen 1220 of a catheter 1212 is attached to a motor 1250 at its proximal end 1226 rather than an RF generator as in FIG. 11. The motor 1250 is configured to rotate the guidewire 1222 around its longitudinal axis. The device can have one or more additional lumen(s), which can optionally have one or more openings on one or more sides of the catheter. The rotation of the guidewire 1222 produces a rotary cutting or cauterizing effect at the distal tip 1230 of the guidewire 1222 which is useful in crossing occlusions, particularly hardened or calcified lesions which are difficult or impossible to cross with a standard guidewire. The guidewire 1222 can be rotated at any speed, but the preferred speed is between about 1000 rpm to about 200,000 rpm. At the preferred speed, the guidewire 1222 has a cutting/cauterizing effect which is not achieved by slower rotation, e.g. hand manipulation. In some embodiments, the speed is, is about, is at least about, is no more than, or is no more than about, 1000, 2000, 3000, 4000, 5000, 6000, 7000, 8000, 9000, 10,000, 15,000, 20,000, 25,000, 50,000, 75,000, 100,000, 150,000, or 200,000 rpm, or falls within a range defined by any two of these values. In a preferred embodiment, a means for stabilizing the rotation of the guidewire is provided.

In another embodiment, the motor 1250 or a similar device is configured to vibrate the distal tip 1230 of the guidewire 1222, instead of or in addition to rotating. The vibration can be at any frequency, but the preferred frequency is about 20 kHz to about 100 kHz. In some embodiments, the frequency is, is about, is at least about, is no more than, or is no more than about, 20, 25, 30, 35, 40, 45, 50, 55, 60, 65, 70, 75, 80, 85, 90, 95, or 100 kHz, or falls within a range defined by any two of these values. The vibration can be in a generally longitudinal or transverse direction along the longitudinal axis of the guidewire, a torsion direction, or combination of both.

In another embodiment, the motor 1250 or similar device is configured to oscillate the distal tip 1230 of the guidewire 1222 around its longitudinal axis, or along the length of the longitudinal axis. The oscillation can be at any frequency, but the preferred frequency is about 10 oscillations per second to about 5000 oscillations per second. In some embodiments, the frequency is, is about, is at least about, is no more than, or is no more than about, 10, 50, 100, 200, 300, 400, 500, 600, 700, 800, 900, 1000, 1500, 2000, 2500, 3000, 3500, 4000, 4500, 5000 oscillations per second, or falls within a range defined by any two of these values. The oscillation around the longitudinal axis can be through any angle, including greater than 365°, but is preferably between about 5° and about 365°, more preferably between about 90° and about 275°. It is contemplated that the oscillation can have an angle of deflection around the longitudinal axis that is, is about, is at least, is at least about, is not more than, is not more than about, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 degrees, or can fall within a range defined by any two of these values. Oscillation along the length of the longitudinal can be over a distance of between about 5 mm and 0.5 mm. However, it is contemplated that the distance is, is about, is at least, is at least about, is not more than, or is not more than about, 0.1, 0.5, 1, 1.5, 2, 2.5, 3, 3.5, 4, 4.5, and 5 mm, or can fall within a range defined by any two of these values.

Because high speed rotation, vibration or oscillation of the guidewire 1222 can pose a greater danger of damage to the blood vessel than slow rotation, vibration, or oscillation, it is preferable to use the rotating, vibrating, and/or oscillating guidewire 1222 under the real-time visual guidance provided by an ultrasound device 1234 because it is safer and more effective. Preferably, the ultrasound imaging device 1234 is configured to provide real-time imaging of the environment in front of the distal tip 1232 of the catheter 1212, i.e. forward-looking. In other configurations, the ultrasound device 1234 can be configured to provide a view of the vessel wall beside the distal end 1214 of the catheter (i.e., side-looking). In other configurations, the device is able to provide a wide field of view, such that it provides both side and forward-looking capabilities. The guidewire 1222 can be used to safely and effectively cross an occlusion of a blood vessel under the visual guidance provided by the ultrasound imaging device 1234. In a preferred embodiment, the IVUS imaging device 1234 comprises the compliant mechanism disclosed herein, for example, as disclosed in FIG. 3, although the use of other ultrasound devices is contemplated.

In one embodiment a support means .1252 is provided on the distal end 1214 of the catheter with a channel 1254 which provides additional stability to the distal end 1224 of the guidewire 1222. The support means 1252 can be a solid cap that has a center channel formed to be slightly larger than the outside diameter of the guidewire 1222. Preferably, the cap is made from a substance, or the inside of the channel is coated with a substance, that reduces friction between the guidewire 1222 and the support means 1252. Similarly, the distal end 1224 and/or tip 1230 of the guidewire 1222 can also be treated to reduce friction between the guidewire 1222 and the support means 1252. In other embodiments, the support means 1252 is a concentric bearing in which the distal end 1224 of the guidewire fits tightly enough that the bearing rotates along with the guidewire 1222. Additional support means can be provided along the length of the catheter as needed.

FIG. 12B is a perspective view of the distal end 1214 of the device shown in FIG. 12A. The support means 1252, distal tip 1230 of the guidewire, and the ultrasound device 1234 are shown.

To further enhance the rotational/vibrational cutting or cauterizing effect of the guidewire, and thereby more efficiently and effectively cross the vascular occlusion, the distal tip 1230 of the guidewire 1222 of FIG. 12 can optionally have a cutting means. The cutting means can be a shape of the distal tip and/or a treatment of the distal tip with an abrasive substance, for example diamond powder.

FIG. 13 illustrates a few of the preferred shapes for the distal tip 1330 of the guidewire: it can be pointed as shown in FIG. 13a, angled as shown in FIG. 13b, or auger shaped as shown in FIG. 13c. FIG. 13d illustrates a distal tip 1330 of a guidewire where the tip is made from a coiled wire. Other shapes, such as a star or concave tip, can be used, as one of skill in the art will recognize.

In some embodiments the guidewire 1212 is attached to one or more devices (not shown) to provide both RF energy and rotation/vibration/oscillatory movement, combining the benefits of both RF energy and rotary/vibrational/oscillatory cutting or cauterizing in a single guidewire. The guidewire 1222 can be configured to provide RF energy and rotational/vibrational/oscillatory energy simultaneously, or sequentially, as is needed. This combination provides a particularly effective method for crossing hardened vascular lesions. In an alternative embodiment not shown, two guidewires are disposed in a single catheter, with one guidewire connected to an RF generator and configured to emit RF energy from the distal tip, (for example, as is disclosed in FIG. 11 and the accompanying text), while the a second guidewire is attached to a motor which rotates, vibrates, and/or oscillates the distal tip of the second guidewire (for example, as is disclosed in FIG. 12 and the accompanying text). Also contemplated is the use of two guidewires to deliver RF energy in a bi-polar configuration where the first guidewire is configured to emit RF energy from the distal tip, (for example, as is disclosed in FIG. 11 and the accompanying text), and the second guidewire, preferably the distal tip, acts as the ground electrode (similar to the wire 1144 shown in FIG. 11, and described in the accompanying text).

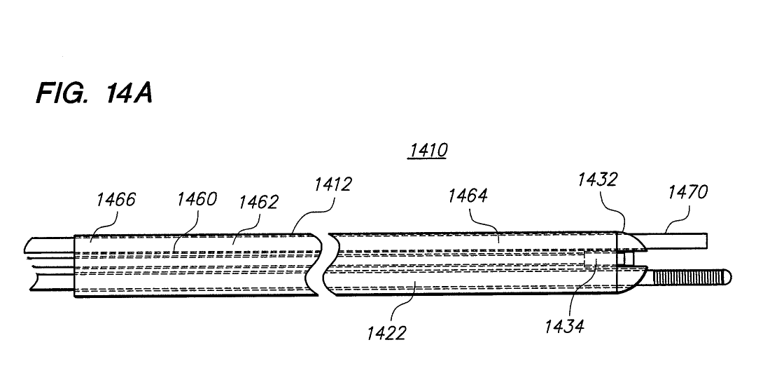

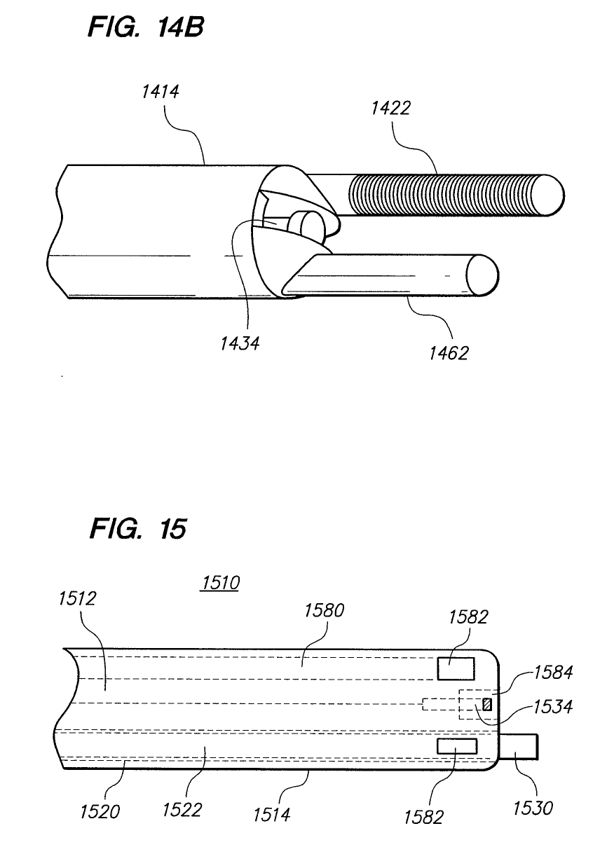

In another embodiment of the invention shown in FIG. 14A, the medical device 1410 has an additional lumen 1460 traversing the longitudinal axis of the catheter 1412. Disposed within the lumen is an optical fiber 1462 which has a distal 1464 and proximal 1466 end. The optical fiber 1462 can optionally be disposed within the lumen 1460 of the catheter 1412 such that the optical fiber 1462 can move axially within the lumen, such that the distal tip 1470 of the optical fiber can be extended past the distal tip 1432 of the catheter 1412. The optical fiber is configured for use in Optical Coherence Tomography (OCT), a technique known to those of skill in the art. Briefly described, OCT uses infrared light delivered through the optical fiber 1462 with a polished prism/mirror at the distal tip 1470. The reflective backscatter is read and computed with respect to the different reflective indices of various cells and tissues present near the distal tip 1470. While the resolution of OCT is much greater than ultrasound (10 μm for OCT versus 100 μm for IVUS), the light penetration is limited to about 2 mm, meaning one can not see through to the adventitia. In addition, OCT is computationally intensive, to the point where real-time imaging is challenging. In addition, OCT cannot “see” through blood. Thus, while OCT is useful for generating detailed information about the blood vessel wall, other characteristics are unsuitable for assisting the operator in navigating the device of the invention through the vascular or assisting in safely and effectively guiding the guidewire through a vascular occlusion. Therefore, while it is contemplated that only the optical fiber 1462 equipped for OCT could be used in combination with the guidewire 1422, in the preferred embodiment shown in FIG. 14, the optical fiber 1462 is used in addition to the IVUS device 1434.

FIG. 14B is a perspective view of the distal end 1414 of the device shown in FIG. 14A. The guidewire 1422, optical fiber 1462 and ultrasound device 1434 are shown.

FIG. 15 illustrates the distal end of another embodiment of a medical device 1510 of the invention. Similar to the devices of FIGS. 11, 12 and 14, the device 1510 comprises a catheter 1512 with a distal end 1514. The catheter 1512 has at least two lumens, 1520 and 1580, traversing the longitudinal axis of the catheter 1512. A guidewire 1522 is disposed within one lumen 1520. The lumens 1520 and 1580 can optionally have one or more openings 1582 on one or more sides of the catheter. These openings 1582 can be used to aspirate the area around the distal tip of the device if suction is applied to the proximal end of the lumen 1520 or 1580. Alternatively, fluid can be delivered through the lumen 1520 or 1580 to the opening. For example, fluids in lumen 1520 can be used to cool the guidewire 1522 (e.g., if the guidewire is RF enabled and/or rotating/vibrating/oscillating as described in FIGS. 11-14, and accompanying text) or the area around the distal tip 1530 of the guidewire 1522, or provide lubrication for a rotating/vibrating/oscillating guidewire 1522. In another embodiment, one or more lumens (not shown) supply fluid or aspiration to the area 1584 surrounding the ultrasound device 1534.

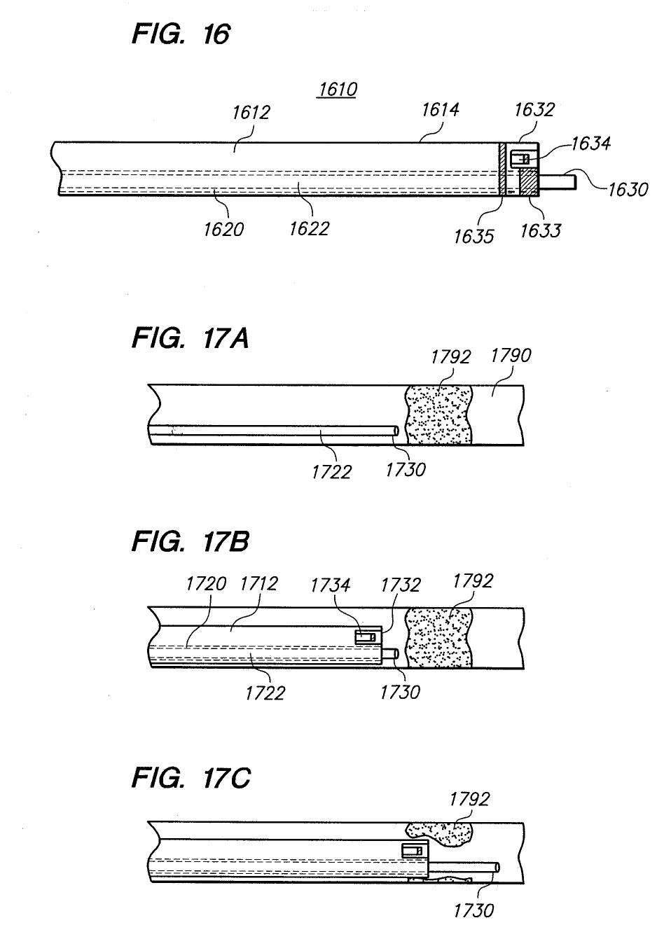

FIG. 16 illustrates the distal end of another embodiment of a medical device 1610 of the invention. Similar to the device of FIG. 11, the device comprises a catheter 1612 with an ultrasound device 1634 located in the distal end 1614 of the catheter 1612. The distal tip 1632 of the catheter 1612 is configured to emit RF energy to assist in crossing difficult lesions, with at least one portion 1633 of the distal tip 1632 of the catheter configured as an RF electrode. In a mono-polar configuration, the patient lies on a grounding pad (not shown). Alternatively, in a bi-polar configuration, the second electrode 1635 is located on the distal end 1614 of the catheter. The electrode(s) are connected to an RF generator at the proximal end of the catheter by a wire(s) traversing the longitudinal axis of the catheter (not shown). The catheter optionally has one or more lumens 1620 traversing the longitudinal axis of the catheter 1612. The lumen(s) can optionally have one or more openings on one or more sides of the catheter. The lumen(s) can be used to aspirate the area around the distal end 1614 of the catheter, provide fluid to one or more portions of the device, etc. as described herein. A guidewire 1622 is optionally disposed within the lumen 1620. In this embodiment, the guidewire 1622 does not act as an electrode for the RF generator. However, it can be equipped for rotary/vibrational/oscillatory cutting as disclosed herein. The distal tip 1632 of the catheter and/or the distal tip 1630 of the guidewire 1622 are advanced through the vascular occlusion under the guidance provided by the ultrasound device 1634. Preferably, the ultrasound device 1634 provides real-time imaging of the area in front of and/or beside the distal end 1614 and/or distal tip 1632 of the catheter 1612.

Because of the small size of the actuator described herein, the IVUS device of the disclosed medical devices can be made extremely small. This allows for the overall dimensions of the disclosed medical devices to be very small as well. In any of the embodiments described herein, the outside diameter of the portion of the device that is placed inside the patient's coronary vasculature is preferably between about 2 Fr -3.5 Fr (about 0.6 mm to about 1.2 mm), although sizes as small as 1 Fr (0.33 mm) are contemplated. For peripheral applications, the outside diameter of the portion of the device placed in the patient's vasculature can be as large as about 12 Fr (4 mm). While these are preferred measurements, it is contemplated that the outside diameter of any portion of the disclosed devices, including the proximal or distal ends, the main body of the device, or the portion of the device designed to be placed inside the patient, is, is about, is not less than, is not less than about, is not more than, or is not more than about 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 mm, or falls within a range defined by any two of these values.

Also contemplated are methods of crossing an occlusion using the devices disclosed herein. In one embodiment, depicted in FIG. 17 A-C, the medical device disclosed herein, e.g. the device of FIGS. 11, 12, 14, 15, or 16, is used to cross an occlusion in the vascular system of an animal, preferably a human. In FIG. 17A, a guidewire 1722 having a distal tip 1730 is threaded through the patient's vasculature 1790 to the point of the occlusion 1792, typically under the guidance of X-ray fluoroscopy. As shown in FIG. 17B, once the guidewire 1722 is in place, a catheter 1712 is threaded over the guidewire 1722, such that the guidewire 1722 is disposed in a lumen 1720 of the catheter 1712. The catheter 1712 is advanced over the guidewire 1722 until the distal tip 1732 of the catheter is near the occlusion 1792. The ultrasound device 1734 is then used to visualize the area in front of and/or beside the distal end of the device, and guide the distal tip 1730 of the guidewire 1722 through the occlusion 1792.

When the guidewire 1722 encounters resistance such that it cannot proceed, the RF generator (not shown) can be used to generate RF energy which is released from the distal tip 1730 of the guidewire 1722 , ablating at least a portion of the occlusion 1792, allowing the guidewire 1722 to be advanced as shown in FIG. 17C. The device can be configured in the monopolar or bipolar configuration, and the polarity of the RF wires can be reversed, as disclosed above (see, e.g., FIGS. 11 and 16 and accompanying text). Repeated RF signals can be generated as needed to cross the occlusion. Alternatively, a motor (not shown) can be used to rotate, vibrate, and/or oscillate the distal tip 1730 of the guidewire 1722 to assist the guidewire 1722 in crossing the occlusion (see, e.g., FIG. 12 and accompanying text). In some embodiments, both RF and rotation/vibration/oscillation of the guidewire 1722, either simultaneously or sequentially, are used to assist in crossing the occlusion 1792. The ultrasound device 1734 is preferably used to safely direct the guidewire 1722 before, during, and/or after the use of the RF and/or rotary/vibrational/oscillatory (kinetic) energy of the guidewire 1722. In an alternative embodiment, the RF energy is emitted from the distal tip 1732 of the catheter 1712, rather than the distal tip 1730 of the guidewire 1722 (see, e.g., FIG. 16 and accompanying text).

As disclosed herein, the catheter 1712 can have one or more additional lumens and/or openings (not shown) to supply fluid to the distal end of the catheter, and/or to aspirate the area around the distal tip 1732 of the catheter 1712. In some embodiments (not shown), the catheter 1712 and guidewire 1722 are advanced simultaneously through the vasculature to the site of the occlusion 1792, optionally under X-ray fluoroscopic guidance, and/or under visual guidance provided by the ultrasound device 1734.

Although the present invention and its advantages have been described in detail, it should be understood that the present invention is not limited to or defined by what is shown or described herein. Known methods, systems, or components may be discussed without giving details, so to avoid obscuring the principles of the invention. As it will be appreciated by one of ordinary skill in the art, various changes, substitutions, and alternations could be made or otherwise implemented without departing from the principles of the present invention. As such, the drawings are for purposes of illustrating a preferred embodiment(s) of the present invention and are not to be construed as limiting the present invention. In particular, it is contemplated that elements and configurations discussed in a particular embodiment can be incorporated in other embodiments, and are not limited to the particular embodiment in which it is described.

What is claimed is:

1. A medical device to cross a vascular occlusion comprising:
an intravascular catheter having a proximal end and distal end, and at least one lumen traversing along the longitudinal axis of the catheter;
a guidewire disposed in said at least one lumen, said guidewire having a proximal end and a distal end, wherein said guidewire can be moved axially within said lumen such that said distal end of said guidewire can be extended beyond the distal end of said catheter;
an ultrasound apparatus having an ultrasound transducer, said ultrasound apparatus located at said distal end of said catheter, wherein said ultrasound apparatus is configured to provide real-time imaging of an area proximate to a distal tip of said catheter;
wherein said device is configured to provide energy to a distal end of said device, wherein said energy is selected from the group comprising radio-frequency energy, rotary energy, vibrational energy, or oscillatory energy; and
wherein said energy is sufficient to assist said device in crossing a vascular occlusion.

2. The medical device of claim 1, further comprising a radio frequency source generator attached to said proximal end of said guidewire, wherein said guidewire is configured to emit radio frequency energy from only a distal tip of said distal end of said guidewire.

3. The medical device of claim 2, wherein said ultrasound device is configured to provide real-time imaging of at least an area in front of said distal tip of said catheter.

4. The medical device of claim 2, wherein said ultrasound device is configured to provide real-time imaging of at least an area at a right angle to the longitudinal axis of the catheter at said distal tip of said catheter.

5. The medical device of claim 2, wherein said device has a bi-polar configuration, and wherein said catheter further comprises an electrode on said distal end of said catheter.

6. The medical device of claim 5, wherein said radio-frequency energy has a frequency of about 200 kHz and about 700 kHz.

7. The medical device of claim 1, wherein said distal tip of said guidewire has a cutting means to facilitate crossing a vascular lesion.

8. The medical device of claim 1, wherein said ultrasound device is configured to provide real-time imaging of at least an area in front of said distal tip of said catheter.

9. The medical device of claim 1, wherein said ultrasound device is configured to provide real-time imaging of at least an area at a right angle to the longitudinal axis of the catheter at said distal tip of said catheter.

10. The medical device of claim 1, further comprising a motor attached to said proximal end of said guidewire, wherein said motor is configured to rotate said guidewire around its longitudinal axis, to vibrate a distal tip of said guidewire, or to oscillate a distal tip of said guidewire.

11. The medical device of claim 10, wherein said motor rotates said guidewire around its longitudinal axis at a rate of about 1000 rpm to about 9000 rpm.

12. The medical device of claim 10, wherein said distal tip of said guidewire has a cutting means to facilitate crossing a vascular lesion.

13. The medical device of claim 12, wherein said ultrasound device is configured to provide real-time imaging of at least an area in front of said distal tip of said catheter.

14. The medical device of claim 12, wherein said ultrasound device is configured to provide real-time imaging of at least an area at a right angle to the longitudinal axis of the catheter at said distal tip of said catheter.

15. The medical device of claim 10, further comprising a radio frequency source generator attached to said proximal end of said guidewire, wherein said guidewire is configured to emit radio frequency energy from only a distal tip of said distal end of said guidewire.

16. The medical device of claim 1, further comprising an optical fiber disposed in an additional lumen, said optical fiber having a proximal end and a distal end, wherein said optical fiber is configured to perform optical coherence tomography.

17. The medical device of claim 2, further comprising a second lumen traversing along the longitudinal axis of the catheter, wherein said second lumen is in communication with the environment at the distal end of said catheter, such that suction applied to the proximal end of said second lumen can be used to aspirate the environment proximate to said distal end of said catheter.

18. The medical device of claim 10, further comprising a second lumen traversing along the longitudinal axis of the catheter, wherein said second lumen is in communication with the environment at the distal end of said catheter, such that suction applied to the proximal end of said second lumen can be used to aspirate the environment proximate to said distal end of said catheter.

19. The medical device of claim 1, further comprising a radio frequency source generator attached to said proximal end of said catheter, wherein said catheter is configured to emit radio frequency energy from only an electrode on said distal tip of said catheter.

20. The medical device of claim 19, wherein said device has a bi-polar configuration, and wherein said catheter further comprises a second electrode on said distal end of said catheter.

21. The medical device of claim 20, further comprising a motor attached to said proximal end of said guidewire, wherein said motor is configured to rotate said guidewire around its longitudinal axis, to vibrate a distal tip of said guidewire, or to oscillate a distal tip of said guidewire.

22. The medical device of claim 21, wherein said ultrasound device is configured to provide real-time imaging of at least the area in front of said distal tip of said catheter.

23. The medical device of claim 2, wherein said ultrasound apparatus comprises a compliant apparatus sized for intravascular use having no mechanical joints and capable of being manipulated through at least one degree of freedom without permanent deformation, the compliant apparatus comprising:
a tubular structure having an axis and formed from a tube made of a material having a reversible structural behavior;
at least one compliant mechanism integrally formed from the tube by removing material from the tube to facilitate bending motion; and
at least one force-generating actuator attached to the compliant apparatus for manipulating the compliant apparatus by bending the at least one compliant mechanism away from the axis;
wherein said ultrasound transducer is coupled to the compliant apparatus.

24. The medical device of claim 10, wherein said ultrasound apparatus comprises a compliant apparatus sized for intravascular use having no mechanical joints and capable of being manipulated through at least one degree of freedom without permanent deformation, the compliant apparatus comprising:
a tubular structure having an axis and formed from a tube made of a material having a reversible structural behavior;
at least one compliant mechanism integrally formed from the tube by removing material from the tube to facilitate bending motion; and
at least one force-generating actuator attached to the compliant apparatus for manipulating the compliant apparatus by bending the at least one compliant mechanism away from the axis;
wherein said ultrasound transducer is coupled to the compliant apparatus.

25. The medical device of claim 20, wherein said ultrasound apparatus comprises a compliant apparatus sized for intravascular use having no mechanical joints and capable of being manipulated through at least one degree of freedom without permanent deformation, the compliant apparatus comprising:
a tubular structure having an axis and formed from a tube made of a material having a reversible structural behavior;
at least one compliant mechanism integrally formed from the tube by removing material from the tube to facilitate bending motion; and
at least one force-generating actuator attached to the compliant apparatus for manipulating the compliant apparatus by bending the at least one compliant mechanism away from the axis;
wherein said ultrasound transducer is coupled to the compliant apparatus.

26. A method for crossing a vascular occlusion comprising:
inserting the distal end of a guidewire having a distal end and a proximal end into the vasculature of an animal having a vascular occlusion;
advancing said distal end of said guidewire through the vascular to said occlusion;
passing the distal end of an intravascular catheter having a proximal end, a distal end, and at least one lumen traversing along the longitudinal axis of the catheter over the proximal end of said guidewire such that said proximal end of said guidewire is disposed in said lumen;
advancing said distal end of said catheter over the guidewire until said distal end of said guidewire is proximate to said occlusion;
using an ultrasound apparatus having an ultrasound transducer, said ultrasound apparatus located at said distal end of said catheter, to provide real-time imaging of the environment proximate to a distal tip of said catheter;
advancing said distal tip of said guidewire through said occlusion under the guidance of said real-time imaging until said guidewire encounters a portion of the occlusion which cannot be crossed; and
providing energy to the distal end of said guidewire wherein said energy is at least radio-frequency, rotational, vibrational, or oscillatory, such that said energy permits the advancement of said distal tip of said guidewire through said occlusion.

27. The method of claim 26, wherein said ultrasound apparatus comprises a compliant apparatus sized for intravascular use having no mechanical joints and capable of being manipulated through at least one degree of freedom without permanent deformation, the compliant apparatus comprising:
a tubular structure having an axis and formed from a tube made of a material having a reversible structural behavior;
at least one compliant mechanism integrally formed from the tube by removing material from the tube to facilitate bending motion;
at least one force-generating actuator attached to the compliant apparatus for manipulating the compliant apparatus by bending the at least one compliant mechanism away from the axis; and
wherein said ultrasound transducer is coupled to the compliant apparatus.

28. The method of claim 27, wherein said energy is radio-frequency energy and said energy is provided by activating a radio-frequency generator attached to said proximal end of said guidewire such that radio-frequency energy is emitted from only a distal tip of said guidewire; and
wherein said radio-frequency energy has a frequency of about 200 kHz and about 700 kHz.

29. The method of claim 27, wherein said energy is rotational, vibrational or oscillatory energy and said energy is provided by activating a motor attached to said proximal end of said guidewire such that a distal tip of said guidewire is rotated, vibrated, or oscillated.

30. The medical device of claim 29, wherein said motor rotates said guidewire around its longitudinal axis at a rate of about 1000 rpm to about 9000 rpm.

31. The method of claim 27, wherein said energy is radio-frequency energy and said energy is provided by activating a radio-frequency generator attached to said proximal end of said catheter such that radio-frequency energy is emitted from an electrode on said distal tip of said catheter; and
wherein said radio-frequency energy has a frequency of about 200 kHz and about 700 kHz.

32. The method of claim 27, wherein said ultrasound device is configured to provide real-time imaging of at least the area in front of said distal tip of said catheter.

33. The medical device of claim 26, wherein said ultrasound device is configured to provide real-time imaging of at least an area at a right angle to the longitudinal axis of the catheter at said distal tip of said catheter.

34. The method of claim 26, wherein said intravascular catheter further comprises an optical fiber disposed in a second lumen, said optical fiber having a proximal end and a distal end, wherein said optical fiber is configured to perform optical coherence tomography; and
using said optical fiber to perform optical coherence tomography to provide real-time imaging of the environment proximate to a distal tip of said catheter.
